(19) United States
(12) Patent Application Publication
(10) Pub. No.: US 2019/0160218 A1
(43) Pub. Date: May 30, 2019

(54) DIALYSIS MACHINE HAVING A CONTROL UNIT FOR CARRYING OUT A CONDITIONING OF THE DIALYSIS MEMBRANE

(71) Applicant: FRESENIUS MEDICAL CARE DEUTSCHLAND GMBH, Bad Homburg (DE)

(72) Inventors: Reiner SPICKERMANN, Wasserlosen-Burghausen (DE); Alfred GAGEL, Litzendorf (DE)

(73) Assignee: FRESENIUS MEDICAL CARE DEUTSCHLAND GMBH, Bad Homburg (DE)

(21) Appl. No.: 16/318,639

(22) PCT Filed: Jul. 18, 2017
(86) PCT No.: PCT/EP2017/000868

§ 371 Date: Jan. 17, 2019

(30) Foreign Application Priority Data
Jul. 18, 2016 (DE) 10 2016 008 755.2

(51) Int. Cl. A61M 1/36; A61M 1/16

(52) U.S. Cl. CPC A61M 1/365; A61M 1/1676; A61M 2202/0057; A61M 2202/0445; A61M 1/1601

(57) ABSTRACT

The present invention relates to a dialysis machine having an extracorporeal blood circuit; a dialyzer; and a dialysate circuit, wherein a blood pump is arranged in the extracorporeal blood circuit; wherein the dialyzer has a dialysate membrane which separates its blood side from its dialysate side; wherein the dialysis machine has a control unit which is configured to carry out a conditioning cycle which comprises a conditioning phase in which an ultrafiltration rate exceeds the blood flow through the dialyzer conveyed by the blood pump.

The invention relates to a dialysis machine having an extracorporeal blood circuit, a dialyzer and a dialysate circuit, wherein a blood pump is arranged in the extracorporeal blood circuit.

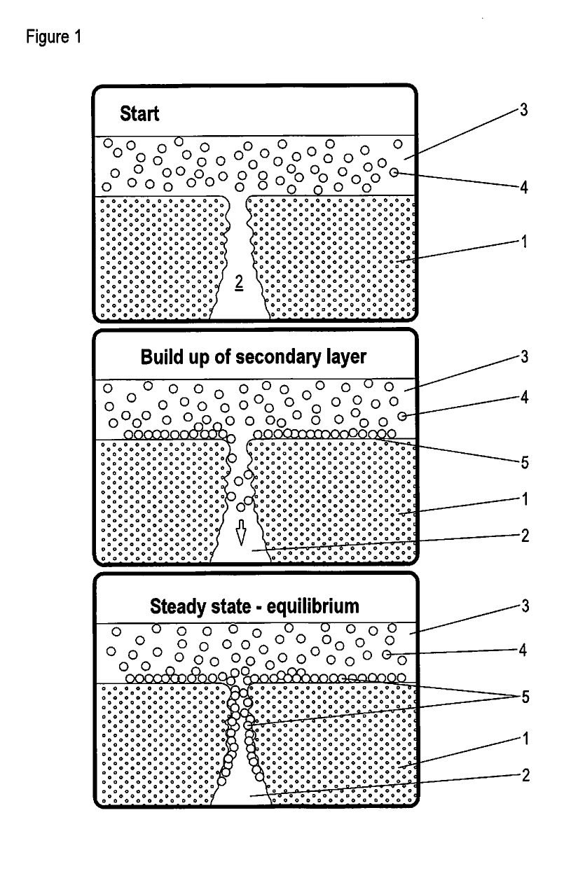

At the start of a dialysis treatment, for example a hemodialysis treatment or a hemodiafiltration treatment, dialyzers show a very high permeability for larger molecules, in particular for albumin. The dialysis membrane is changed by the contact with specific blood components, in particular blood proteins, so that the screening effect for large proteins such as albumin is highly improved. In detail, a secondary membrane which produces a desired better screening effect for large proteins such as albumin is formed by the deposition of albumin and other blood components on the dialysis membrane. This change process is shown schematically in FIG. 1, with the upper image showing the membrane 1 with membrane pores 2 at the start of the treatment. In an early phase of the treatment, the plasma proteins 4 in the blood 3 then build up a secondary layer 5 about the pore 2 and in the pore 3 (middle image) until a steady state is reached (lower image).

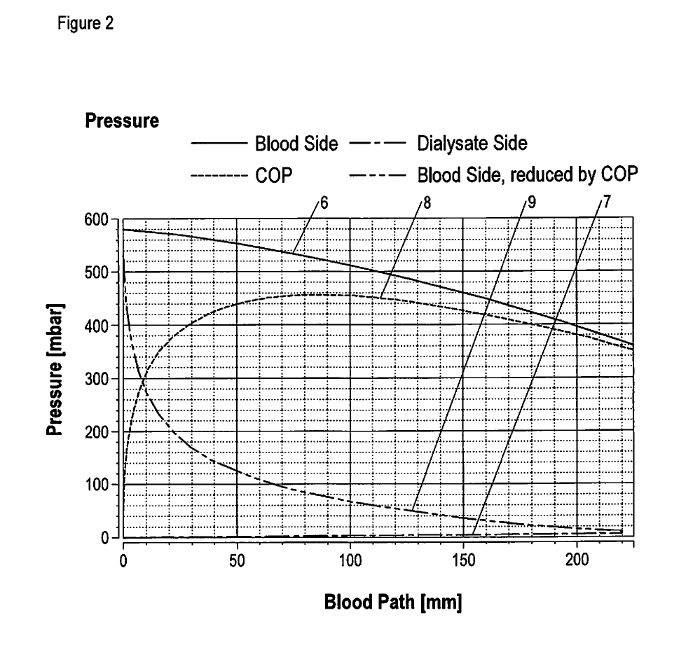

The specification of the above-described membrane conditioning in HDF therapies, for example, is determined by the level and the duration of the substitution rate. The higher the exchange rate, the better and faster the conditioning of the membrane develops. However, the local filtration performance along the dialyzer membrane differs greatly because the local transmembrane pressure (TMP) and the local oncotic pressure vary. This can be recognized in FIG. 2 which shows extents of the hydrostatic pressures at the blood side (p_{B}, reference numeral 6) and at the dialysate side (p_{D}, reference numeral 7), of the oncotic pressure (COP; reference numeral 8) and of the effective pressure (p_{B}-COP; reference numeral 9) along the dialyzer membrane.

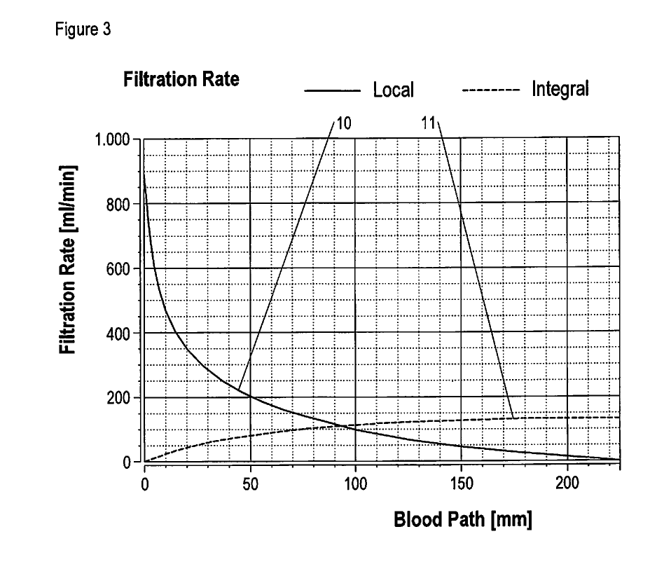

While the local filtration at the blood-side inlet of the dialyzer is very high, it drops fast along the dialyzer or along the capillaries and tends toward zero or can even become negative (in this case, a back filtration is spoken of) at the outlet of the dialyzer. The extents of the local filtration at the blood-side inlet (reference numeral 10) and of the integral filtration over the covered distance at the dialyzer membrane (reference numeral 11) are shown in FIG. 3. Correspondingly large differences are to be expected in, for example, the speed and quality of the conditioning of the filter in accordance with FIG. 1.

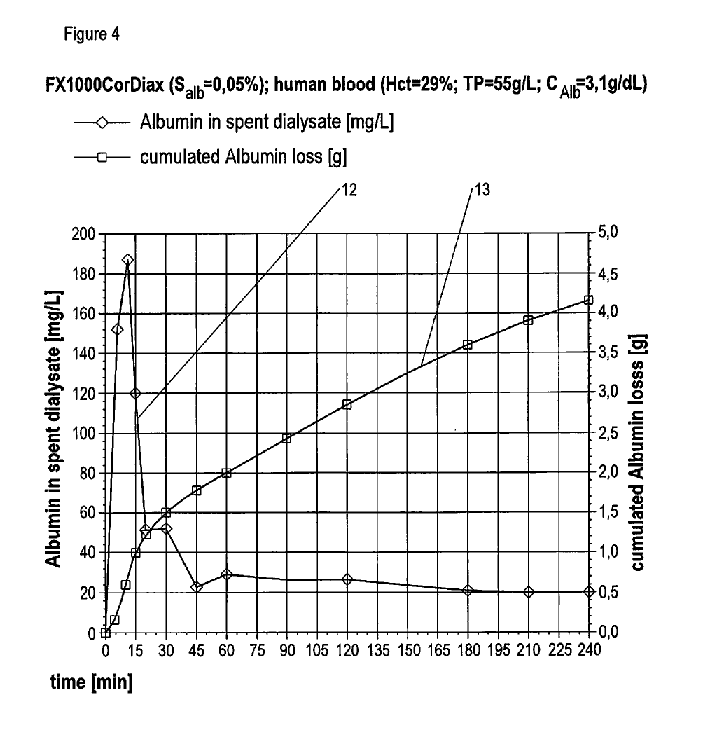

The region (close to the outlet) of the filter, which has only been moderately conditioned under certain circumstances, can cause a relatively high contribution to the albumin loss during the treatment time, even if the exchange volume is small in this region. In the present case, albumin loss overall means the loss of plasma proteins of which albumin is, however, the largest and most important portion. Reference is therefore made to this as representative in the present case. This can be recognized in FIG. 4 where measured temporal extents of the instantaneous albumin concentration (reference numeral 12) and of the cumulated albumin concentration (reference numeral 13) in the dialysate are shown.

It is the object of the invention to provide a dialysis machine with which it is achieved that the local screening coefficients are reduced more uniformly at all points of the membrane.

Against this background, the invention relates to a dialysis machine having an extracorporeal blood circuit, a dialyzer and a dialysate circuit, wherein the dialyzer has a dialysis membrane which separates its blood side from its dialysate side. Provision is made in accordance with the invention that the dialysis machine has a control unit which is configured to carry out a conditioning cycle which comprises a conditioning phase in which an ultrafiltration rate exceeds the blood flow through the dialyzer which is conveyed by the blood pump. A pressure gradient is therefore produced over the dialysis membrane which is accompanied by a liquid withdrawal from the blood.

The ultrafiltration rate can be regulated, for example, with reference to an ultrafiltration pump. Alternatively or additionally, other actuators such as substitution pumps can also intervene in the regulation. The ultrafiltration rate is generally understood in the present case as the rate of the liquid withdrawal.

The dialysis machine has a blood pump which is arranged in the extracorporeal blood circuit and which regulates the blood flow. The blood pump can be located in the arterial branch of the extracorporeal blood circuit.

The ultrafiltration rate during the conditioning phase can correspond to or differ from a normal ultrafiltration rate, i.e. an ultrafiltration rate also present within the framework of the treatment. It can, for example, amount to between 30 ml/min and 150 ml/min, and preferably between 50 ml/min and 100 ml/min. Example values comprise 60 ml/min and 90 ml/min.

The pressure differences over the length of the dialyzer are now only small due to the small flow rate so that an approximately uniform or at least more uniform application of blood proteins and in particular albumin takes place on the membrane over the length of the dialyzer. The initially described membrane conditioning thus takes place more uniform or the secondary membrane is formed uniformly. If this process starts at the beginning of a dialysis, the albumin loss can in particular be significantly reduced at the start of the treatment in which the membrane is normally not conditioned or is only irregularly conditioned.

The dialyzer can be a capillary dialyzer.

In an embodiment, the conditioning cycle is carried out at the start of the treatment. At the start of the treatment and on the carrying out of the conditioning cycle, the blood circuit is already completely filled with blood or the dialysis machine is already connected to the patient. The dialysate circuit is furthermore also filled with liquid at the start of the conditioning cycle.

The (dialysis) treatment can, for example, be a hemodialysis treatment or a hemodiafiltration treatment.

In an embodiment, the ultrafiltration rate exceeds the blood flow through the dialyzer by a specific measure during the conditioning phase. For example, the ratio of the ultrafiltration rate in the dialyzer to the blood flow rate can be between 1.5 to 1 and 2.5 to 1, preferably between 1.8 to 1 and 2.2 to 1, and preferably approximately 2 to 1. A thickening of the blood then takes place in the dialyzer and backflow of blood from the venous line. The pressure differences in the dialyzer are small, which results in a uniform filtration over the length of the dialyzer.

Provision is made in an embodiment that little or no dialysate flows into the dialysate side of the dialyzer during the conditioning phase. The inflow rate can, for example, amount to less than 1000 ml/min or less than 300 ml/min. The inflow corresponds to the standard process of hemofiltration. To achieve a more uniform layer in the conditioning phase, a pressure gradient which is as small as possible is also desirable over the length of the dialyzer on the dialysate side so that the local ultrafiltration rates along the dialyzer are of the same size whenever possible. This is achieved in this embodiment at a low or disabled dialysate flow. Only, or substantially only, the ultrafiltrate is then withdrawn at the dialyzer outlet.

In an embodiment, the duration of the conditioning phase amounts to 15 to 120 seconds and preferably to 30 to 60 seconds.

In an embodiment, a liquid volume of between 20% and 70%, and preferably of between 30% and 60%, of the total volume of the blood side of the dialyzer is withdrawn through the dialysis membrane during the conditioning phase. A temporary thickening of the blood hereby takes place in the dialyzer. Provision can, for example, be made that ½ or ⅓ of the total volume on the blood side of the dialyzer is withdrawn through the dialysis membrane.

The total volume on the blood side of the dialyzer can be between 50 and 200 ml and preferably between 80 and 150 ml. Example values include 95 ml, 115 ml or 136 ml. These values are related to the size of the filter. The fluid volume which is withdrawn during the conditioning phase can be between 15 and 80 ml, and preferably between 25 and 70 ml. These values depend on the flow rates of the pumps and on the transmembrane pressure derived therefrom.

The transmembrane pressure can also be used to regulate substitution rates and, indirectly, the blood flow rate. On an exceeding of a limit value for the transmembrane pressure, precautions can be taken for a reduction or a shutdown can take place.

In an embodiment, the conditioning cycle is repeated, preferably at least 3× and, for example, between 3× and 6×. Provision is preferably made that the repeats follow directly on from one another. The conditioning of the membrane can thereby be further improved. If a plurality of conditioning cycles are provided which preferably follow one another, they can all take place at the treatment start.

In an embodiment, the conditioning cycle comprises a flushing phase which follows the conditioning phase and in which the blood flow through the dialyzer exceeds the ultrafiltration rate. Provision can, for example, be made that the ultrafiltration pump is switched off in this phase or is operated at a rate which is reduced or is the same with respect to the conditioning phase. The thickened blood is washed out of the dialyzer in this phase. The flushing phase preferably starts directly after the conditioning phase. A further layer build-up can thus take place by fresh blood and a constant thickening is avoided which would result in clotting.

In an embodiment, the blood volume used in the flushing phase to flush the blood chamber of the dialyzer can amount to 0.5 to 5 times, and preferably 1 to 2 times, the blood chamber volume.

In an embodiment, the conditioning cycle only comprises the conditioning phase and the flushing phase. Measurements can be made during the phases or between the phases.

In an embodiment, the dialysis machine has a substitution line which preferably leads into the venous line of the extracorporeal blood circuit, with the control unit being configured such that the fluid volume withdrawn in the conditioning phase in the dialyzer is at least partly substituted using this line, preferably in the conditioning phase and/or during the flushing phase.

The idea of the invention furthermore comprises a method for carrying out a dialysis treatment at a machine in accordance with the invention, wherein a conditioning cycle is carried out such as was described in accordance with the machine in accordance with the invention.

It can be stated that a uniform design of a secondary membrane can be achieved over the membrane surface of the dialyzer using the present invention. A secondary membrane can furthermore be formed faster. The albumin losses can be minimized initially and cumulatively in a subsequent dialysis treatment.

The hemocompatibility can furthermore be increased. For if blood comes into contact with foreign surfaces, a variety of reactions can occur. If a secondary membrane is, however, built up fast and uniformly as in the present invention, the blood in the dialyzer only comes into contact with the blood's own substances at larger surface regions in the dialyzer.

In addition, the loss of substances which should not be removed, for example the loss of enzymes or hormones, is reduced by the reduction of the screening coefficient.

Further details and advantages of the invention result from the Figures and embodiments discussed in the following. There are shown in the Figures:

FIG. 1: a schematic representation of the change process when the dialysis membrane comes into contact with blood proteins in an early treatment stage;

FIG. 2: extents of the hydrostatic pressures on the blood side and on the dialysate side, of the oncotic pressure and of the effective pressure along the dialyzer membrane;

FIG. 3: extents of the local filtration at the blood-side inlet and of the integral filtration over the path covered at the dialyzer membrane;

FIG. 4: time progressions of the instantaneous albumin concentration and of the cumulated albumin concentration in the dialysate;

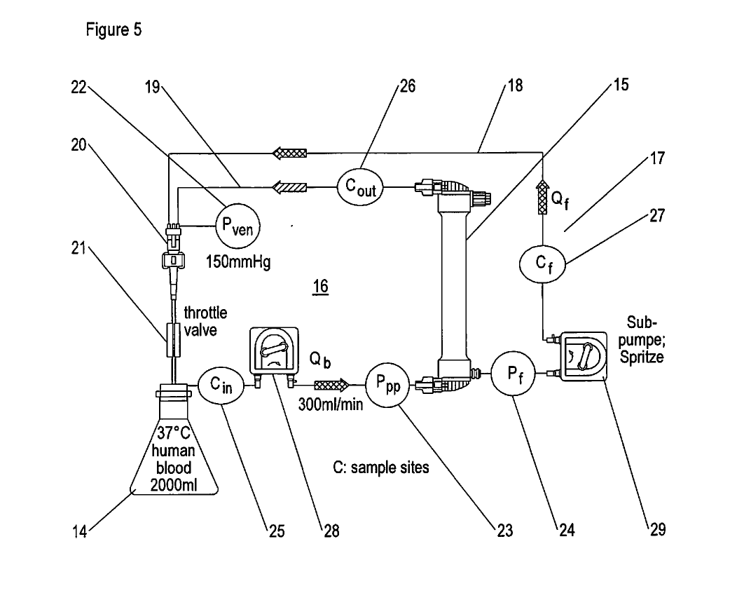

FIG. 5: an experimental setup for the experimental testing of the invention;

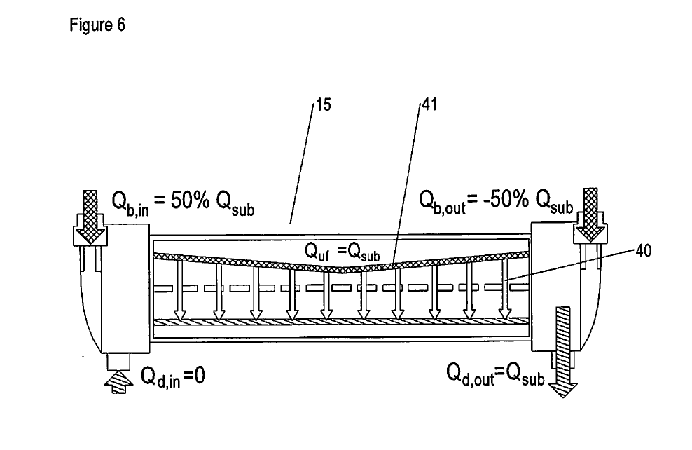

FIG. 6 a schematic representation of the flow progressions and of the pressure progressions in the dialyzer at a ratio of the ultrafiltration rate to the blood flow rate of 2 to 1;

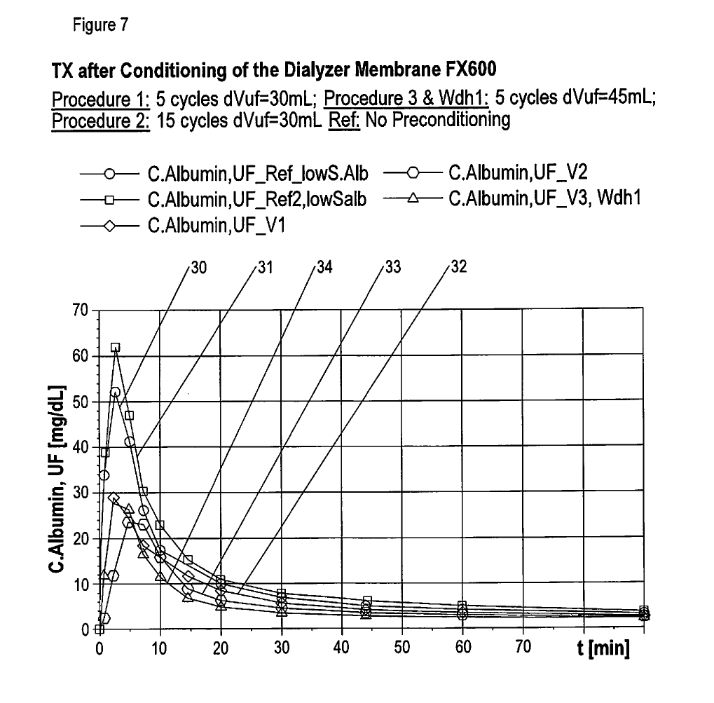

FIG. 7: a graphical representation of the time progressions of determined albumin concentrations in the ultrafiltrate; and

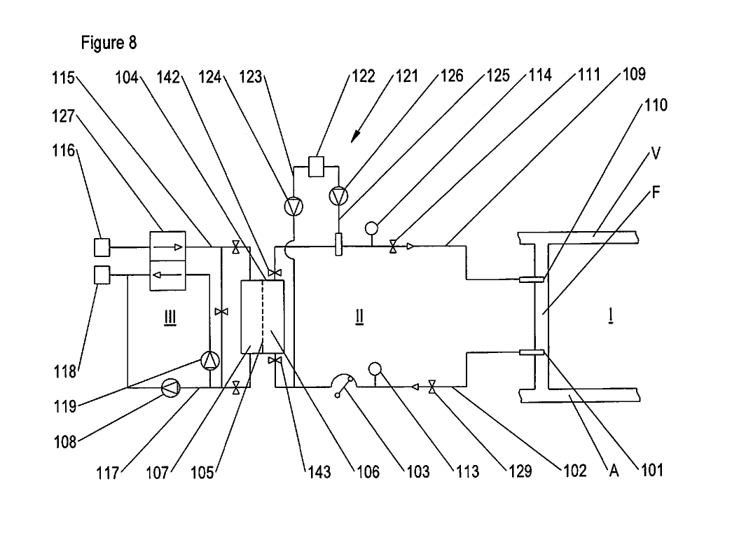

FIG. 8: a schematic representation of a dialysis machine in accordance with the invention.

An experimental setup for the experimental testing of the invention is shown in FIG. 5. A vessel is marked by reference numeral 14 in which blood can be located at a body temperature of approximately 37° C. in a volume of, for example, 2000 ml. A dialyzer is marked by reference numeral 15; a simulated extracorporeal blood circuit by reference numeral 16; and a simulated dialysate circuit by reference numeral 17. In the experimental arrangement, the dialysate circuit 17 having the outflow line 18 for the substitution of the dialysate is subsequent to the venous line 19 of the blood circuit 16, and admittedly at a drip chamber 20. A throttle valve 21 is located between the drip chamber 20 and the vessel 14.

Pressure sensors 22, 23 an 24 serve the determination of the pressures Pᵥₑₙ, P_{PP} and P_{f}. Reference numerals 25, 26 and 27 mark measurement points for the concentrations Cᵢₙ, Cₒᵤₜ and C_{f}. A blood flow Q_{b} of, for example, 300 ml/min can be produced using the blood pump 28 arranged downstream of the dialyzer 15 in the blood circuit 16. A dialysate flow or a substitute flow Q_{f} can be produced using the ultrafiltration pump 29 arranged downstream of the dialyzer 15 in the dialysate circuit 17, with the it simultaneously being a substitution pump or syringe in the experimental setup.

The venous hose system 19 has to be connected to the (“patient”) volume free of air so that a volume flow of, for example, 40 ml/min can be withdrawn over the venous branch. The pressure Pᵥₑₙ can amount, for example, to 150 mmHg.

The following experiments were carried out with this arrangement. The following parameters were approximately identical in all experiments:

Dialyzer type: FX600 (V_{b}=95 mL)

Screening coefficient: S_{alb}=0.032%

Blood flow: Q_{b}=300 ml/min

Hematocrit: Hkt=36%±2%

Albumin: C_{alb}≥3.0 g/dL

### EXAMPLE 1

The experimental setup was first prepared in accordance with FIG. 5 (step 1). The dialyzer 15 was filled and flushed with NaCl solution at the blood side and at the dialysate side and residual air was completely removed at both sides of the dialyzer 15 by knocking. The blood side was subsequently filled with blood (step 3).

In accordance with the invention, the blood pump 28 was then stopped (step 4) to reduce pressure drops along the capillaries of the dialyzer 15.

Approximately ⅓ of the volume on the blood side was withdrawn over the membrane using a substitution pump 29 (step 5). The value for ΔV_{uf} amounted to 30 ml. The ultrafiltration volume ΔV_{uf} is thus drawn uniformly over the membrane. The procedure was as follows: The substitution pump 29 draws at the dialysate side at Q_{uf}=60 ml/min until the desired ultrafiltration (UF) volume of 30 ml per cycle has been withdrawn (duration approximately 30 s to 60 s). The blood pump 28 runs in this phase at half the UF rate at Q_{b}=30 ml/min. The residual volume flow (approximately 30 ml/min) is taken back over the venous hose system 19.

The ratio of the UF rate Q_{uf} (60 ml/min to the lead flow rate of the blood Q_{b,in} (30 ml/min) amounts to exactly 2/1 in this example. FIG. 6 schematically shows the flow development 40 and the pressure drop 41 in the dialyzer 15 which results at such a ratio. Half of the flow Q_{d,out} of the filtrate withdrawn from the blood, which corresponds exactly to the ultrafiltration rate Q_{uf} at a dialysate inflow rate Q_{d,in} of 0 ml/min, comes from the arterial line where the blood pump 28 resupplies blood. Since the blood chamber of the dialyzer 15 is openly connected to the venous line 19, blood is also sucked in from the venous line 19 (30 ml/min). Pressure conditions which are as uniform as possible are achieved for a layer which is as uniform as possible by the symmetrical distribution. The pressure gradient on the blood side of the dialyzer is thus the lowest.

Subsequently a determination of the albumin loss in the ultrafiltration volume takes place using the formula Δm_{alb}=C_{alb}*ΔV_{uf} (step 6). The withdrawn sample ultrafiltration volume ΔV_{uf} is again led back into the storage vessel 14.

The blood pump 28 is then started again at Q_{b}=300 ml/min (step 7). The thickened blood is thus flushed out of the dialyzer 15 again.

After 2 min (600 ml blood volume), the blood pump 28 is stopped again (step 8).

After step 8, either the conditioning cycle of steps 5 to 8 can be repeated or a transition to step 9 can take place. In the present embodiment, the conditioning cycle of steps 5 to 8 is run through a total of 5 times; that is, the total V_{uf} in the conditioning cycle amounts to 150 ml and a total of 5 samples are taken.

In the final step 9, a post HF treatment is simulated having a substitution rate of Q_{sub}=100 ml/min and a duration of T=180 min. Samples are taken from the ultrafiltrate (C_{f}, measurement point 25) and from the storage vessel (Cᵢₙ; measurement point 27) at test times (t=0 min; t=2 min 30 sec; t=5 min; t=7 min 30 sec; t=10 min; t=15 min; t=20 min; t=30 min; t=45 min; t=60 min; t=90 min; t=120 min; t=150 min; t=180 min) and the undisturbed pressures Pᵥₑₙ, P_{PP} and P_{f} are noted.

The evaluation detected (a) the albumin loss and myoglobin loss during the conditioning phase, i.e. during the 5 times repetition of steps 5 to 8; and (b) the albumin loss during the 3-hour treatment.

### EXAMPLE 2

This example was largely identical to Example 1, the conditioning cycle, i.e. steps 5 to 8 were, however, repeated 15 times, that is, the total V_{uf} in the conditioning cycle amounts to 450 mL and the procedure of step 5 was as follows: The substitution pump 29 draws at the dialysate side at Q_{uf}=90 ml/min until the desired ultrafiltration (UF) volume of 30 ml per cycle has been withdrawn (duration approximately 30 s to 60 s). The blood pump 28 runs in this phase at half the UF rate at Q_{b}=45 ml/min. The residual volume flow (approximately 45 ml/min) is taken back over the venous hose system 19.

The evaluation took place as in Example 1.

In accordance with a modified Example 2′, a setting of Q_{b} at 40 ml/min would also be conceivable, which would result in a return flow rate from the venous line of 50 ml/min at an ultrafiltration rate Q_{uf}=90 nil/min.

### EXAMPLE 3

This example was also largely identical to Example 1, but here approximately ½ of the volume on the blood side was withdrawn over the membrane with the syringe or substitution pump 29 in step 5, that is, ΔV_{uf}=45 ml, with a 5× running through of the conditioning cycle of steps 5 to 8, that is 225 m.

The evaluation again took place as in Example 1.

### COMPARISON EXAMPLE 4

After carrying out steps 1 to 3 in accordance with Example 1, steps 4 to 8 were omitted and a post-HF treatment was simulated as in step 9 with a substitution rate of Q_{sub}=100 ml/min.

The evaluation again took place as in Example 1.

The same experiments as in Examples 1 to 4 could also be carried out using alternative dialyzers, for example with an FX800 (V_{b}=115 ml) or an FX1000 (V_{b}=136 ml). In Example 1, step 5, the ultrafiltration volume ΔV_{uf} would then amount to 37 ml (FX800) or 44 ml (FX1000) and with a 5 time repetition of the conditioning cycle a total ΔV_{uf} of 185 ml (FX800) or 220 ml (FX1000) would result. In Example 2, that is, with a 15 time repetition of the conditioning cycle this would result in a total ΔV_{uf} of 555 ml (FX800) or 660 ml (FX1000). In Example 3, the ultrafiltration volume ΔV_{uf} would then amount to 55 ml (FX800) or 65 ml (FX1000) and with a 5 time repetition of the conditioning cycle a total ΔV_{uf} of 275 ml (FX800) or 325 ml (FX1000) would result.

The absolute and relative albumin losses and myoglobin losses measured for Examples 1 to 4 over the total treatment time (determined in the ultrafiltrate at measurement point 27) are shown in Table 1.

TABLE 1

| | | | Myoglobin | Myoglobin |
|---|---|---|---|---|
| | Albumin loss, | Albumin loss, | loss, | loss, |
| Example | absolute | relative | absolute | relative |
| V4 | 1.08 g | | 22.3 mg | |
| V4 | 1.24 g | | 20.7 mg | |
| (double) | | | | |
| 1 | 0.85 g | −26% | 21.2 mg | −1% |
| 2 | 0.77 g | −33% | 21.2 mg | −1% |
| 3 | 0.68 g | −41% | 20.3 mg | −6% |

The time progressions of the albumin concentrations in the ultrafiltrate determined at the measurement point 27 are shown in FIG. 7, wherein reference numeral 30 shows the curve for comparison example 4; reference numeral 31 shows the curve for the double of comparison Example 4; reference numeral 32 shows the curve for Example 1; reference numeral 33 shows the curve for Example 2; and reference numeral 34 shows the curve for Example 3.

The averages of the transmembrane pressures TMP measured over the treatment time and differences P_{PP}−Pᵥₑₙ are shown in Table 2.

TABLE 2

| Example | TMP | P_{PP}-Pᵥₑₙ |
|---|---|---|
| V4 | | |
| V4 (double) | 190 mmHg | 161 mmHg |
| 1 | 150 mmHg | 150 mmHg |
| 2 | 174 mmHg | 161 mmHg |
| 3 | 160 mmHg | 158 mmHg |

In summary, it can be stated with reference to the examples that the albumin losses during the conditioning cycle were negligible due to the small exchanged volumes (ΔV_{uf}=5*30 ml=150 ml; C_{alb,max}=0.5 mg/ml; Δm_{alb}=75 mg). Example 3 delivered the best conditioning with a value for ΔV_{uf} of 5*45 ml (50% of the blood volume) and with a reduction of the albumin loss by 41%. The conditioning cycle lasts approximately 2 to 3 min per repetition including flushing; that is 10 to 15 min with a 5-fold repetition.

FIG. 8 shows a schematic representation of a dialysis machine in accordance with the invention.

In FIG. 8, the blood system of the patient is marked by reference numeral I; the extracorporeal blood circuit of the machine by reference numeral II; and the dialysate side of the machine by reference numeral III.

The principle in the sense of the invention can be implemented at such a machine using the experiment setup described in more detail above.

The blood system I of the patient comprises an artery A, a vein V and a fistula F.

The extracorporeal blood circuit is connected to the fistula using the needles 101 and 110. The arterial line 102 which has a blood pump 103 and opens downstream into the dialyzer 104 adjoins the arterial needle 101. The venous line 109 runs to the venous needle 110 from the dialyzer 104. The extracorporeal blood circuit comprises arterial and venous pressure sensors 113 and 114 as well as clamps 129, 143, 142 and 111.

The dialyzer 104 comprises a dialyzer membrane 105 by which the blood chamber 106 is separated from the dialysate chamber 107.

The dialysate system III comprises a dialysate source 116. a balancing device 127 and an infeed 115 which leads into the dialysate chamber 107 of the dialyzer 104. The ultrafiltration pump and dialysis fluid pump 108 and 119 are arranged in a drain line 117 from the dialysate chamber 107. The return is marked by the reference numeral 118.

A substitution fluid system 121 comprises a substitution fluid source 122 as well as a predilution line and predilution pump 123 and 124 and a post-dilution line and post-dilution pump 125 and 126.

The dialysis machine shown, unlike FIG. 5, does not only represent an experimental setup, but rather a real machine in accordance with the invention at which the inventive principle can be used.

1. A dialysis machine having an extracorporeal blood circuit, a dialyzer, and a dialysate circuit, wherein a blood pump is arranged in the extracorporeal blood circuit; and wherein the dialyzer has a dialysis membrane which separates its blood side from its dialysate side,
characterized in that the dialysis machine has a control unit which is configured to carry out a conditioning cycle which comprises a conditioning phase in which an ultrafiltration rate exceeds the blood flow through the dialyzer which is conveyed by the blood pump.

2. A dialysis machine in accordance with claim 1, characterized in that the conditioning cycle is carried out at the start of the treatment.

3. A dialysis machine in accordance with claim 1, characterized in that the ratio of the ultrafiltration rate to the blood flow rate caused by the blood pump amounts to between 1.5 to 1 and 2.5 to 1, preferably between 1.8 to 1 and 2.2 to 1, and preferably approximately 2 to 1.

4. A dialysis machine in accordance with claim 1, characterized in that little or no dialysate flows into the dialysate side of the dialyzer during the conditioning phase.

5. A dialysis machine in accordance with claim 1, characterized in that the duration of the conditioning phase amounts to 15 to 120 seconds, and preferably 30 to 60 seconds.

6. A dialysis machine in accordance with claim 1, characterized in that a fluid volume of between 20% and 70%, and preferably of between 30% and 60%, of the total volume of the blood side of the dialyzer is withdrawn through the dialysis membrane during the conditioning phase.

7. A dialysis machine in accordance with claim 1, characterized in that the conditioning cycle is repeated, preferably at least 3×, and, for example, between 3× and 6×, with the repetitions preferably directly following one another.

8. A dialysis machine in accordance with claim 1, characterized in that the conditioning cycle comprises a flushing phase which follows the conditioning phase and in which the blood flow through the dialyzer generated by the blood pump exceeds the ultrafiltration rate.

9. A dialysis machine in accordance with claim 8, characterized in that the blood volume used in the flushing phase to flush the blood chamber of the dialyzer amounts to 0.5 to 5 times, and preferably 1 to 2 times, the blood chamber volume.

10. A dialysis machine in accordance with claim 1, characterized in that the dialysis machine has a substitution line which preferably leads into the venous line of the extracorporeal blood circuit, with the control unit being configured such that the fluid volume withdrawn in the conditioning phase in the dialyzer is at least partly substituted using this line, preferably after the conditioning phase and during the flushing phase.
